(19) United States
(12) Patent Application Publication
(10) Pub. No.: US 2022/0296903 A1
(43) Pub. Date: Sep. 22, 2022

(54) METHODS AND SYSTEMS FOR LONG TERM TREATMENT OF NEUROPSYCHIATRIC DISORDERS

(71) Applicant: Magnus Medical, Inc., Burlingame, CA (US)

(72) Inventors: Adi MARON-KATZ, Binyamina-Giv'at Ada (IL); Brandon S. BENTZLEY, Half Moon Bay, CA (US); Brett M. WINGEIER, San Francisco, CA (US)

(21) Appl. No.: 17/700,451

(22) Filed: Mar. 21, 2022

Related U.S. Application Data
(60) Provisional application No. 63/164,261, filed on Mar. 22, 2021.

(51) Int. Cl. A61N 1/36; A61B 5/00

(52) U.S. Cl. CPC A61N 1/36096; A61N 1/36025; A61B 5/4076

(57) ABSTRACT

Described herein are systems and methods for the automated prediction of relapse of a neurological or psychiatric disorder. The systems and methods may generally use patient data such as patient characteristics, treatment history, clinical history, biometric data, and/or neuroimaging data as inputs to a predictive model. Additionally, the systems and methods may be integrated with a treatment system so that neurostimulation may be automatically delivered when triggered by the predictive model. Systems and methods configured to propose a personalized treatment schedule for maintaining the effects of neurostimulation therapy are also described herein.

### CROSS-REFERENCE TO RELATED APPLICATIONS

This application claims priority to U.S. Provisional Application Ser. No. 63/164,261, filed on Mar. 22, 2021, which is hereby incorporated by reference in its entirety.

### FIELD

This application generally relates to the treatment of a neurological or a psychiatric disorder. More specifically, the application relates to systems and methods for the prediction of relapse in patients with a neurological or a psychiatric disorder. The systems and methods may employ an algorithm that automates the prediction of relapse. Systems and methods for the determination of maintenance treatment of neurological or psychiatric disorders (e.g., depression) with transcranial magnetic stimulation or another treatment modality are also described herein.

### BACKGROUND

Depression, including major depressive disorder (MDD), bipolar disorder (BD), and peripartum depression (PPD), is the leading cause of disability worldwide and is characterized by a high rate of recurrence. Epidemiological and clinical evidence suggests that major depressive disorder typically follows a recurrent course, with a third to a half of patients relapsing within one year of discontinuation of treatment, and that a greater number of prior depressive episodes is associated with a higher probability of future recurrence. After treatment of the first episode of depression, approximately half of all patients will relapse, and the risk tends to increase for every subsequent episode. Therefore, it may be beneficial to identify the warning signs of relapse early in order to prevent relapse and improve the overall disease trajectory (Sim K, Lau W K, Sim J, Sum M Y, Baldessarini R J. Int J Neuropsychopharmacology, 2015, 1-13; Moriarty A S, Castleton J, Gilbody S, McMillan D, Ali S, Riley R D, Chew-Graham C A. Br J Gen Prac, 2020, 54-55).

Daily monitoring of warning signs of relapse would be ideal. However, it may be impractical to complete a daily clinician administered inventory, such as the Montgomery-Asberg Depression Scale (MADRS) or Hamilton Rating Scale for Depression (HAM-D), given the time and specialization required to administer them. Thus, the development of systems and methods that use readily available information, such as biosignals and quick, patient-reported inventories, for early detection and/or prediction of relapse may be useful in facilitating early detection and/or prediction of relapse.

Adequate treatment of depression is hindered by the lack of methods to detect or predict relapse. Standard antidepressants can be effective for treatment of MDD. However, many patients do not respond to these medications (treatment-resistant depression, or TRD), and many patients desire to reduce or end their chronic use of antidepressants. Acute interventions may also be used to treat depression as described below; additionally, many patients are in need of acute treatment for depression due to suicidality or hospitalization. When acute treatments are delivered or medication is reduced or terminated, many patients relapse (e.g., within 6 months of apparent clinical response or remission), with faster and higher rates of relapse observed in those with treatment-resistant depression (TRD). However, rapid-acting antidepressant, subanesthetic, intravenous (IV) ketamine has demonstrated efficacy in maintaining antidepressant effect beyond the acute treatment period. Additionally, long-term maintenance therapy regimens have shown promise in prevention of relapse (Sing J B, Fedgchin M, Daly E J, Drevets W C, Adv Pharmacol, 2020, 89:237-259). However, ketamine is a drug of abuse and has serious side effects. Therefore, it would be beneficial to administer the therapy only when needed (on-demand). The ability to trigger therapy after early detection and/or prediction of relapse would reduce the exposure to ketamine.

Recently, psychedelics have shown some effectiveness as a therapy for TRD (Davis A K, Barrett F S, May D G, et al. JAMA Psychiatry 2021, 78(5):481-489). Currently, the use of psychedelics requires dosing and monitoring by a psychotherapist. This model may be time and therapist intensive (Nutt D, Carhart-Harris R, JAMA Psychiatry, 2020E1-E2). Additionally, psychedelics are scheduled as a very dangerous drug. Thus, it would be beneficial to deliver psychedelics only after early detections and/or prediction of relapse.

Transcranial Magnetic Stimulation (TMS) is a non-invasive medical procedure where strong magnetic fields are utilized to stimulate specific areas of an individual's brain in order to treat medical conditions such as depression and obsessive-compulsive disorder (OCD). When TMS is repeatedly applied in a short time frame, it is referred to as repetitive TMS (rTMS). Theta-burst stimulation (TBS) is a patterned form of rTMS, typically administered as a triplet of stimulus pulses with 20 ms between each stimulus in the triplet (therefore having a pulse frequency of 50 Hz), where the triplet is repeated every 200 ms (therefore having triplets, or bursts, occurring at a frequency of 5 Hz), although other combinations of pulse and burst timing may also be used.

Acute rTMS therapy is an approved and acknowledged treatment for MDD (Perera T, George M S, Grammer G, et al. Brain Stimulat 2016, 9:336-346; Milev R V, Giacobbe P, Kennedy S K, et al. Can J Psychiatry Rev Can Psychiatr 2016, 61:561-575) and has been shown to achieve significant antidepressant effects (Sehatzadeh S, Daskalakis Z J, Yap B, Tu H A, Palimaka S, Bowen J M, O'Reilly D J. J Psychiatry Neurosci, 2019, 44:151-163). Acute rTMS therapy has demonstrated similar response and remission rates compared to antidepressant medication therapy alone (monotherapy) as well as psychotherapy plus antidepressants (Baeken C, Brem A-K, Arns M, et al. Curr Opin Psychiatry, 2019, 32:409-415).

Although acute therapy may be successful, patients with depression, especially treatment-resistant depression (TRD), may have a high relapse rate, with relapse occurring weeks or months after acute TMS treatment. Also, depression may be episodic, and a patient who has responded or remitted after acute TMS treatment may relapse by entering a new episode of depression months or years after acute TMS treatment. In patients who have responded previously to TMS therapy, re-treatment with TMS therapy has been shown to be effective when relapse occurs. However, waiting for relapse is undesirable because the symptoms of depression must be experienced again before re-treatment occurs.

Maintenance rTMS therapy (that is, re-treatment with rTMS therapy without requiring that relapse has fully occurred) for patients with depression may be undertaken using fixed maintenance schedules (for instance, one session or day of maintenance treatment per month, or one week of sessions per six-month period). The development of maintenance rTMS therapy may effectively reduce or prevent the relapse of depression, and decrease the overall burden of depression symptoms, in depression patients who initially responded to acute rTMS treatment (Chang J, Chu Y, Ren Y, Li C, Wang Y, Chu Z-P, Int J Physiol Pathophysiol Pharmacol 2020; 12(5): 128-133).

However, maintenance TMS therapy using a fixed maintenance schedule is not generally used in part due to the cost, inconvenience, and potential exposure to side effects of “unnecessary” scheduled treatment in cases where relapse is not actually impending. The fact that such TMS maintenance protocols are not generally used after acute TMS treatment has led to response or remission may be one of the reasons for the relapse rate observed in TRD patients.

Accordingly, it would be useful to have systems and methods that predict relapse and initiate treatment when treatment is needed. The automatic delivery of treatment when relapse is predicted would also be useful.

### SUMMARY

Described herein are systems and methods for the prediction of relapse of a neurological or a psychiatric disorder. The systems may include machine learning, and run algorithms that use patient data such as patient characteristics, treatment history, clinical history, biometric data, neuroimaging data, or a combination thereof, as inputs to generate a predictive model. The predictive model may predict patient relapse with minimal clinician intervention. Additionally, the systems and methods may be integrated with a treatment system so that neurostimulation may be automatically delivered when triggered by the predictive model. Systems and methods configured to propose a personalized treatment schedule based on the data inputs from the patient for maintaining the effects of neurostimulation therapy are also described herein.

The systems may be used to predict relapse of various psychiatric disorders such as, but not limited to, depression, treatment-resistant depression, anxiety, post-traumatic stress disorder (PTSD), obsessive-compulsive disorder (OCD), a substance use disorder, bipolar disorder, or schizophrenia. Exemplary neurological disorders in which the systems may be used to predict relapse include without limitation, Parkinson's disease, essential tremor, stroke, epilepsy, traumatic brain injury, migraine headache, cluster headache, or chronic pain.

The systems for predicting relapse of a neurological or a psychiatric disorder of a patient may generally include a device configured to obtain one or more data features from the patient and a data module. The data module may comprise one or more processors configured to run a machine learning algorithm, where the machine learning algorithm may be configured to analyze the one or more data features, generate a mood report based on the analyzed one or more data features, generate a mood plot having a mood threshold predetermined for the patient based on a plurality of mood reports taken over a plurality of neurostimulation treatment sessions, and predict relapse of the neurological or the psychiatric disorder in the patient based on the mood plot.

When the onset of relapse is predicted, the system may be configured to issue an alert or other warning signal that notifies the patient and/or the clinician of the relapse, and that treatment, for example, maintenance treatment, is needed. The alert may be an audible alarm, a visual alarm, a text, an email, or a combination thereof. In some instances, the system may include a treatment device, for example, a transcranial magnetic stimulation (TMS) device, that may be automatically triggered or manually activated to deliver neurostimulation therapy upon receipt of the prediction of relapse.

In general, the methods for predicting relapse of a neurological or a psychiatric disorder of a patient may include inputting one or more data features from the patient into a predictive model for the neurological or the psychiatric disorder, and applying a machine learning algorithm to the one or more data features to generate a mood report and a mood plot, where the mood plot has a mood threshold predetermined for the patient based on a plurality of mood reports taken over a plurality of neurostimulation treatment sessions. Relapse of the neurological or the psychiatric disorder in the patient may then be predicted based on the mood plot.

The methods described herein may be used to predict relapse of various psychiatric disorders such as, but not limited to, depression, treatment-resistant depression, anxiety, post-traumatic stress disorder (PTSD), obsessive-compulsive disorder (OCD), a substance use disorder, bipolar disorder, or schizophrenia. Exemplary neurological disorders in which the methods may be used to predict relapse include without limitation, Parkinson's disease, essential tremor, stroke, epilepsy, traumatic brain injury, migraine headache, cluster headache, or chronic pain.

When neurostimulation is to be delivered by the system, delivery may be automatically triggered, or manually activated, as mentioned above. The neurostimulation may be generated by a TMS device, such as a TMS coil, and delivered according to a treatment schedule. The treatment schedule may be a personalized treatment schedule recommended by the machine learning algorithm of the system based on the data inputs obtained from the patient.

### BRIEF DESCRIPTION OF THE DRAWINGS

FIG. 1 is a flowchart of an exemplary method for predicting relapse based on patient-reported mood reports and previous treatment data.

FIG. 2. is a flow chart describing another exemplary method for predicting the score on a standard assessment of a neurological or psychiatric disorder, and triggering therapy.

FIG. 3 is a flowchart of an exemplary method for determining a set of potential predictors for model test data.

FIG. 4 is a flowchart of an exemplary method for determining outcomes for model training data.

FIG. 5 is a flowchart of an exemplary method for implementing a machine learning approach to extract features and develop a set of regression models.

FIG. 6 depicts an example of the topology of a decision tree created during Feature of Importance evaluation by Random Forest Classification.

FIG. 7 is a flowchart of an exemplary method for implementing a machine learning approach to extract features and develop a set of classification models.

FIG. 8 is a flowchart of an exemplary method for predicting relapse and sending an alert to a clinician to start maintenance therapy.

FIG. 9 illustrates an exemplary feature selection procedure.

FIG. 10 depicts an exemplary decision tree model for predicting relapse.

FIGS. 11A and 11B illustrate the performance of an exemplary feature selection procedure.

FIGS. 12A and 12B illustrate a clinical example of relapse prediction.

### DETAILED DESCRIPTION

Described herein are systems and methods for the prediction of relapse for a patient with a neurological or psychiatric disorder. The prediction may be based on various data features processed by a machine learning algorithm, and thus may be automated. If relapse is predicted, the systems and methods may be generally configured to trigger re-treatment of the neurological or psychiatric disorder.

Psychiatric disorders that may be treated include without limitation, depression, treatment-resistant depression, anxiety, post-traumatic stress disorder (PTSD), obsessive-compulsive disorder (OCD), addictions, substance use disorders such as opioid, stimulant, tobacco, or alcohol use disorders, bipolar disorder, and schizophrenia. Neurological disorders and associated symptoms that may be treated include without limitation, Parkinson's disease, essential tremor, stroke, epilepsy, traumatic brain injury, migraine headache, cluster headache, chronic pain.

### Systems

The machine learning systems described herein generally analyze data and establish models to make predictions, e.g., predictions of relapse of a neurological or a psychiatric disorder. Examples of machine learning tasks may include classification, regression, and clustering. A predictive engine may be a machine learning system that includes a data processing framework and one or more algorithms trained and configured based on collections of data.

The systems described herein may generally include a device configured to obtain one or more data features from the patient and a data module. The device for obtaining the one or more data features may comprise a computer, a laptop, a tablet computer, a mobile phone, a smart-watch, a ring configured to collect data features, or an implantable or partially-implanted device such as an implant dedicated to collection of data features or a neurostimulation implant additionally configured to collect data features. The data module may comprise one or more processors configured to run a machine learning algorithm, where the machine learning algorithm may be configured to analyze the one or more data features, generate a mood report based on the analyzed one or more data features, generate a mood plot having a mood threshold predetermined for the patient based on a plurality of mood reports taken over a plurality of neurostimulation treatment sessions, and predict relapse of the neurological or the psychological disorder in the patient based on the mood plot. Relapse may be predicted if the machine learning algorithm determines that the mood plot does not meet the predetermined mood threshold for the patient.

The machine learning algorithm may comprise a random forest, a boosted decision tree, a classification tree, a regression tree, a bagging tree, a neural network, or a rotation forest. The machine learning algorithm may be, for example, support vector machines, linear regression, logistic regression, linear discriminant analysis, decision trees, k-nearest neighbor algorithm, neural networks, similarity learning, or a combination thereof. In some variations, the parameters of the machine learning algorithm may be adjusted with the aid of a clinician and/or computer system.

The machine learning algorithm may process or analyze one or more data features obtained from the patient. For example, the machine learning algorithm may process data that may be relevant for detecting relapse of a neurological or a psychiatric disorder. In one variation, the one or more data features includes mood data. The mood data may be a patient self-report of daily mood using a visual analog scale (100 to −100), where the best possible mood is scored 100 and the worst possible mood is scored −100. The patient may use a mobile application that consists of a sliding scale where the nominal value of the scale is 0. In another variation, the mood data includes psychometric data. Examples of psychometric data include without limitation, information relating to mind wandering, anxiety, processing speed, task switching ability, attention, loneliness, or a combination thereof. Additionally or alternatively, the one or more data features may include information relating to motor activity.

In some variations, the machine learning algorithm may be a support vector machine trained, e.g., on previous patient data, and may be used to analyze a patient's data and determine whether the patient is: (a) not likely to relapse in a period of time, e.g., 24 hours; or (b) likely to relapse in a period of time. The period of time may range from about 4 hours to about one week, including all values and sub-ranges therein. For example, the time period may be about 4, about 5, about 6, about 7, about 8, about 9, about 10, about 11, about 12, about 13, about 14, about 15, about 16, about 17, about 18, about 19, about 20, about 21, about 22, about 23, or about 24 hours; or about 2, about 3, about 4, about 5, about 6, or about 7 days. The time period may also be less than 4 hours or greater than one week. In some variations, the machine learning algorithm may be used to additionally determine (c) whether the patient is already in a state of relapse.

In other variations, the one or more data features may comprise information related to heart rate, heart rate variability, electroencephalography, electrogastrography, electrogastroenterography, galvanic skin response, sleep, sweat chloride, neuroimaging, patient demographics, outcome data from an acute treatment, outcome data from a prior maintenance treatment, or a combination thereof. The information related to sleep may include a total duration of sleep, a sleep onset time, a sleep offset time, a sleep cycle duration, a number of sleep cycles per night, sleep movements, sleep vocalizations, or a combination thereof. In further variations, the one or more data features may include body temperature or fluctuation in body temperature, such as standard deviation of body temperature. The one or more data features may also include information estimated from a clinician administered inventory.

The systems may be configured to predict when a patient is beginning to relapse into the active disease state, for instance, relapse to a depressive episode, and/or to propose an individualized or personalized treatment schedule that may maximize the likelihood of keeping the patient in remission or response. In some variations, the system may include a component configured to provide an alert to the patient and/or their treatment team that an immediate intervention is required to reduce the risk of relapse to a depressive episode. The alert may be provided between about 4 hours to about one week before the patient relapses, including all values and sub-ranges therein. For example, the alert may be provided about 4, about 5, about 6, about 7, about 8, about 9, about 10, about 11, about 12, about 13, about 14, about 15, about 16, about 17, about 18, about 19, about 20, about 21, about 22, about 23, or about 24 hours before the patient relapses; or about 2, about 3, about 4, about 5, about 6, or about 7 days before the patient relapses. In some instances, the alert may be provided fewer than 4 hours or more than one week before the patient relapses.

The system may also be configured to prospectively recommend a treatment schedule to minimize relapse, e.g., based on outcomes from acute treatment (such as timing and magnitude of response or remission), patient characteristics (such as age, weight, gender), neuroimaging data (such as degree of frontal hypoactivity and/or subgenual cingulate hyperactivity before and/or after treatment), or other data features or combinations of the data features described above.

In some variations, the system may be configured to recommend a preliminary maintenance schedule including, e.g., timing and number of days of treatment, after acute treatment is complete, e.g., using a support vector machine or other classification algorithm operating on data features such as patient characteristics and acute outcome data. The support vector machine or classification algorithm may assign the patient to one of two or more categories, for example: (a) no maintenance schedule needed; (b) maintenance needed once per six months; (c) maintenance needed once per month; or (d) maintenance needed once per week.

The system may include a data collection facility or device such as a computer, a laptop, a tablet computer, a mobile phone, a smart-watch, or a wearable device such as a ring or patch, having a mobile application that collects actigraphy or other data features from the patient and that presents a mood inventory at intervals. In some variations, presenting a mood inventory comprises asking the patient to answer one or more questions regarding mood or other symptoms of depression such as anxiety. In additional variations, questions regarding mood may be presented via means other than a screen or display interface, such as by signaling the patient to answer using sound or vibration, and/or by the patient responding by tapping, turning, or otherwise moving the wearable device. The collected data may be transmitted to the system via a wired or wireless connection.

Based on these data and/or other data, such as patient characteristics or outcomes from previous maintenance treatment, the system may then periodically (e.g., once per day) reclassify the patient as likely or unlikely to relapse in various time intervals, and may notify the patient or a clinician if the patient is classified as likely to relapse in a given time interval (e.g., in the next 24 hours). Based on these data, the system may also recommend an adapted maintenance schedule, e.g., a more frequent or less frequent maintenance schedule. In other variations, the system may be configured to learn or adapt from its experience with one patient to others, or from its experience at one point in time with one patient to a later point in time with the same patient, e.g., using a support vector machine or other algorithm.

The systems described herein may also include a treatment device. An exemplary treatment device may be configured to deliver neurostimulation therapy. In one variation, the treatment device comprises a transcranial magnetic stimulation coil configured to deliver transcranial magnetic stimulation (TMS). Other forms of neuromodulation may also be employed. In some variations, the treatment delivered may include medication, psychotherapy, adjustment or activation of an implantable neurostimulator, or other interventions.

When the systems include a treatment device configured to deliver magnetic neurostimulation therapy, the magnetic stimulation may be accelerated theta-burst stimulation (aTBS), such as accelerated intermittent theta-burst stimulation (aiTBS) or accelerated continuous theta-burst stimulation (acTBS), delivered transcranially according to the SAINT (Stanford Accelerated Intelligent Neuromodulation Therapy) protocol. The SAINT protocol may include applying iTBS pulse trains for multiple sessions per day, for several days. In one variation, the SAINT protocol may include the delivery of neurostimulation for five days. More specifically, the neurostimulation may be delivered for 10 sessions a day, with each session lasting 10 minutes, and an intersession interval (the interval between sessions) of 50 minutes.

The stimulation frequency of the TBS pulses may range from about 20 Hz to about 70 Hz, including all values and sub-ranges therein. For example, the stimulation frequency may be about 20 Hz, about 25 Hz, about 30 Hz, about 35 Hz, about 40 Hz, about 45 Hz, about 50 Hz, about 55 Hz, about 60 Hz, about 65 Hz, or about 70 Hz. When iTBS is used, the burst frequency (that is, the reciprocal of the period of bursting, for example if a burst occurs every 200 ms the burst frequency is 5 Hz) of the iTBS pulses may range from about 3 Hz to about 7 Hz, including all values and sub-ranges therein. For example, the burst frequency may be about 3 Hz, about 4 Hz, about 5 Hz, about 6 Hz, or about 7 Hz.

The patient may undergo multiple treatment sessions per day. In some variations, the number of treatment sessions per day may range from 2 sessions to 40 sessions. For example, the number of treatment sessions may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40. The number of sessions for iTBS may range from 3 to 15 sessions per day. When cTBS is employed, the number of sessions may range from 10-40 sessions per day. The sessions may be performed on consecutive or non-consecutive days.

Additionally, the duration of the intersession interval may vary and range from about 25 minutes to about 120 minutes, including all values and sub-ranges therein. For example, the intersession interval may be about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, or about 120 minutes.

In variations using iTBS, the pulse parameters may include 3-pulse trains with 50 Hz pulses at a burst frequency of 5 Hz for 2 second trains, with trains every 10 seconds for 10 minute sessions (1,800 total pulses per session). In some variations, the iTBS schedule may include conducting 10 sessions per day with 50 minute intersession intervals for 5 consecutive days (18,000 pulses per day, and 90,000 total pulses).

When cTBS is used, pulse trains may range from about 4 seconds to about 45 seconds, including all values and sub-ranges therein. For example, the pulse train may be about 4 seconds, about 5 seconds, about 10 seconds, about 15 seconds, about 20 seconds, about 25 seconds, about 30 seconds, about 35 seconds, about 40 seconds, or about 45 seconds. In one cTBS variation, the pulse parameters may include 3-pulse trains with 50 Hz pulses at a burst frequency of 5 Hz for 40 second sessions (600 total pulses per session). In another variation, the cTBS pulse parameters may include 3-pulse trains with 30 Hz pulses at a burst frequency of 6 Hz for 44 second sessions (800 total pulses per session). In many cTBS variations, 30 sessions may be applied per day with 15-minute intersession intervals for 5 consecutive days (18,000 pulses per day, 90,000 total pulses).

It is understood that the pulse parameters and schedules used for maintenance treatment may be varied. For example, the number of pulses or frequency of sessions may be increased or decreased depending on the refractoriness of the neurological or psychiatric disorder.

### Methods

The methods described herein for predicting relapse of a neurological or a psychiatric disorder of a patient may include inputting one or more data features from the patient into a predictive model for the neurological or the psychiatric disorder, and applying a machine learning algorithm to the one or more data features to generate a mood report and a mood plot, where the mood plot has a mood threshold predetermined for the patient based on a plurality of mood reports taken over a plurality of neurostimulation treatment sessions. Relapse of the neurological or the psychiatric disorder in the patient may then be predicted based on the mood plot. In some variations, and as further described in Example 2, relapse may be predicted if the machine learning algorithm determines that the mood plot does not meet the predetermined mood threshold for the patient.

The methods may be used to predict relapse of various psychiatric disorders such as, but not limited to, depression, treatment-resistant depression, anxiety, post-traumatic stress disorder (PTSD), obsessive-compulsive disorder (OCD), a substance use disorder, bipolar disorder, or schizophrenia. Exemplary neurological disorders in which the methods may be used to predict relapse include without limitation, Parkinson's disease, essential tremor, stroke, epilepsy, traumatic brain injury, migraine headache, cluster headache, or chronic pain.

As previously described, the machine learning algorithm may comprise a random forest, a boosted decision tree, a classification tree, a regression tree, a bagging tree, a neural network, or a rotation forest. The machine learning algorithm may be, for example, support vector machines, linear regression, logistic regression, linear discriminant analysis, decision trees, k-nearest neighbor algorithm, neural networks, similarity learning, or a combination thereof. In some variations, the parameters of the machine learning algorithm may be adjusted with the aid of a clinician and/or computer system.

The machine learning algorithm may process or analyze one or more data features obtained from the patient. The data features may be input into the predictive model via devices such as a computer, a laptop, a tablet computer, a mobile phone, or a smart-watch. The machine learning algorithm may process data that may be relevant for detecting relapse of a neurological or a psychiatric disorder.

In one variation, the one or more data features includes mood data. The mood data may be a patient self-report of daily mood using a visual analog scale. In another variation, the mood data includes psychometric data. Examples of psychometric data include without limitation, information relating to mind wandering, anxiety, processing speed, task switching ability, attention, loneliness, or a combination thereof. Additionally or alternatively, the one or more data features may include information relating to motor activity. In some variations, the machine learning algorithm may be support vector machine trained, e.g., on previous patient data, and may be used to analyze a patient's data and determine whether the patient is: (a) not likely to relapse in a period of time, e.g., 24 hours; or (b) likely to relapse in a period of time. The period of time may range from about 4 hours to about one week, including all values and sub-ranges therein. For example, the time period may be about 4, about 5, about 6, about 7, about 8, about 9, about 10, about 11, about 12, about 13, about 14, about 15, about 16, about 17, about 18, about 19, about 20, about 21, about 22, about 23, or about 24 hours; or about 2, about 3, about 4, about 5, about 6, or about 7 days. The time period may also be less than 4 hours or greater than one week. In some variations, the machine learning algorithm may be used to additionally determine (c) whether the patient is already in a state of relapse.

In other variations, the one or more data features may comprise information related to heart rate, heart rate variability, electroencephalography, electrogastrography, electrogastroenterography, galvanic skin response, sleep, sweat chloride, neuroimaging, patient demographics, outcome data from an acute treatment, outcome data from a prior maintenance treatment, or a combination thereof. The information related to sleep may include a total duration of sleep, a sleep onset time, a sleep offset time, a sleep cycle duration, a number of sleep cycles per night, sleep movements, sleep vocalizations, or a combination thereof. In further variations, the one or more data features may include body temperature or fluctuation in body temperature, such as standard deviation of body temperature. The one or more data features may also include information estimated from a clinician administered inventory.

The methods may include predicting when a patient is beginning to relapse into the active disease state, for instance, relapse to a depressive episode, and/or to propose an individualized or personalized treatment schedule that may maximize the likelihood of keeping the patient in remission or response. In some variations, the methods may provide an alert to the patient and/or their treatment team that an immediate intervention is required to reduce the risk of relapse to a depressive episode. The alert may be provided between about 4 hours to about one week before the patient relapses, including all values and sub-ranges therein. For example, the alert may be provided about 4, about 5, about 6, about 7, about 8, about 9, about 10, about 11, about 12, about 13, about 14, about 15, about 16, about 17, about 18, about 19, about 20, about 21, about 22, about 23, or about 24 hours before the patient relapses; or about 2, about 3, about 4, about 5, about 6, or about 7 days before the patient relapses. In some instances, the alert may be provided fewer than 4 hours or more than one week before the patient relapses.

The methods described herein may also prospectively recommend a treatment schedule to minimize relapse, e.g., based on outcomes from acute treatment (such as timing and magnitude of response or remission), patient characteristics (such as age, weight, gender), neuroimaging data (such as degree of frontal hypoactivity and/or subgenual cingulate hyperactivity before and/or after treatment), or other data features or combinations of the data features described above.

In some variations, the methods may include recommending a preliminary maintenance schedule including, e.g., timing and number of days of treatment, after acute treatment is complete, e.g., using a support vector machine or other classification algorithm operating on data features such as patient characteristics and acute outcome data. The support vector machine or classification algorithm may assign the patient to one of two or more categories, for example: (a) no maintenance schedule needed; (b) maintenance needed once per six months; (c) maintenance needed once per month; or (d) maintenance needed once per week.

The methods may employ a data collection facility or device such as a computer, a laptop, a tablet computer, a mobile phone, a smart-watch, or a wearable device such as a ring or patch, having a mobile application that collects actigraphy or other data features from the patient and that presents a mood inventory at intervals. In some variations, presenting a mood inventory comprises asking the patient to answer one or more questions regarding mood or other symptoms of depression such as anxiety. In additional variations, questions regarding mood may be presented via means other than a screen or display interface, such as by signaling the patient to answer using sound or vibration, and/or by the patient responding by tapping, turning, or otherwise moving the wearable device. The collected data may be transmitted to the system via a wired or wireless connection.

Based on these data and/or other data, such as patient characteristics or outcomes from previous maintenance treatment, the patient may then periodically (e.g., once per day) be reclassified as likely or unlikely to relapse in various time intervals, and the patient or a clinician notified if the patient is classified as likely to relapse in a given time interval (e.g., in the next 24 hours). Based on these data, an adapted maintenance schedule may be recommended, e.g., a more frequent or less frequent maintenance schedule.

The methods described herein may also employ a treatment device. An exemplary treatment device may be configured to deliver neurostimulation therapy. In one variation, the treatment device comprises a transcranial magnetic stimulation coil configured to deliver transcranial magnetic stimulation (TMS). Other forms of neuromodulation may also be used. In some variations, the treatment delivered may include medication, psychotherapy, adjustment or activation of an implantable neurostimulator, or other interventions.

When neurostimulation is to be delivered by the system, delivery may be automatically triggered, or manually activated, as mentioned above. The neurostimulation may be generated by a TMS device, such as a TMS coil, and delivered according to a treatment schedule. The types and parameters for neurostimulation may be those described above. The treatment schedule may be a personalized treatment schedule recommended by the machine learning algorithm of the system based on the data inputs obtained from the patient.

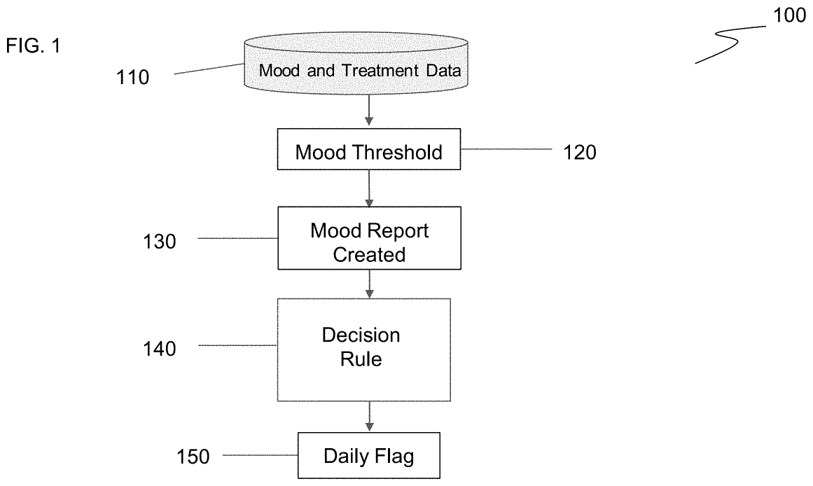

The methods described herein may be generally used to predict relapse of a neurological or psychiatric disorder (e.g., depression) in a patient after initial treatment. In some variations of the treatment schedule, a patient may be treated first with ten sessions of aiTBS (e.g., using the SAINT protocol), and then evaluated for remission after that day. Treatment may then be continued for up to five consecutive days with daily evaluation until remission is achieved. For example, referring to FIG. 1, a flow chart (100) is provided illustrating an exemplary method for predicting relapse. In FIG. 1, the method may comprise: a step (110) of obtaining mood and TMS treatment data from a database, where the mood data is collected from a patient daily by an electronic device; a step (120) of calculating a mood threshold based on a minimum and maximum mood reported up to 30 days post-TMS treatment; and a step (130) of calculating mood report statistics (e.g., t-test and rank-sum test) based on the data slope estimated in a sliding window of 3-6 reports since the last treatment and over the past 21 days, wherein slope estimation can be done, for example, using robust linear regression. Mood plots may then be generated with TMS treatment sessions indicated and a summary report created (for clinician and/or patient). If relapse is predicted by Decision Rule (140), wherein Decision Rule (140) learns by an algorithm taking into account clinician feedback and is initially configured using information describing the individual treatment response of a patient, a Daily Flag (150) may be sent (e.g., via text, email, or other form of notification) to the clinician that indicates recommendation for retreatment, or recommendation for a clinical evaluation to further examine the need for retreatment. In some variations, in addition to or instead of predicting relapse, the Decision Rule (140) predicts the score of a clinician administered inventory such as the MADRS, or predicts whether the score on a clinician administered inventory is above or below a given score, for example, a threshold associated with relapse (such as a score of 10).

In some variations, a report that provides a recommendation for a clinician to further examine the need for retreatment may be generated if one of the following applies (and 9 days have passed since the last TMS treatment): (a) two or more consecutive mood reports below the patient's individually-determined mood threshold (e.g., as calculated by step 120), or (b) the data slope is significantly negative in one or more of the statistics generated (e.g., as calculated by step 130). The report may be used by the clinician to make a decision about re-treatment. If the patient is deemed by the clinician to require re-treatment, the patient may be treated until remission.

As previously mentioned, the mood data may be a patient self-report of daily mood using a visual analog scale (100 to −100), where the best possible mood is scored 100 and the worst possible mood is scored −100. The patient may use a mobile application that consists of a sliding scale wherein the nominal value of the scale is 0. The result of the daily self-report may be uploaded to a database, such as database (110).

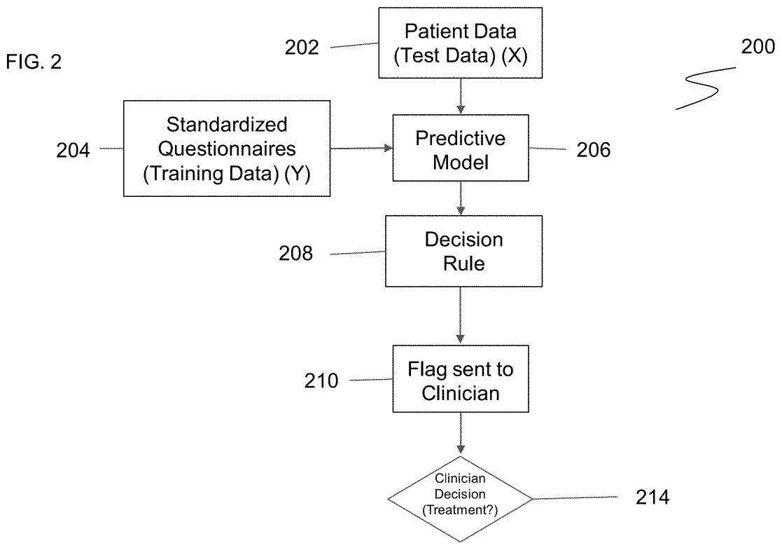

In some variations, the method for predicting relapse of a neurological or psychiatric disorder (such as depression) includes triggering re-treatment. For example, as shown in the flowchart (200) of FIG. 2, a predictive model may be designed to estimate a clinician-administered inventory that is used to assess severity of a neurological or psychiatric disorder. The predictive model may be designed to estimate the likelihood that a clinician administered inventory is above or below a threshold, such as a threshold associated with relapse. A clinician may use one or more inventories as part of the treatment decision-making process. Collection of such inventories generally requires specialized training and may be time consuming. Therefore, it is not practical to administer inventories daily or weekly. Thus, it may be beneficial to use readily accessible information (e.g., patient data) to estimate clinician administered inventories. Patient data including, but not limited to biometrics, patient characteristics, clinical history, and treatment history may be collected or accessed from a database.

Referring to FIG. 2, the method may comprise: a step (202) of obtaining patient data from a database where test data (X) is generated to use in the predictive model, a step (204) of obtaining clinician administered and/or patient-reported inventories from a database, where the inventories are used to generate training data (Y) for use in the predictive model; a step (206) of implementing a predictive model that may include decision-making based on binary thresholds, machine learning algorithms, other methods and/or a combination of methods to create the model; and a step (208) of predicting relapse using a Decision Rule, wherein a Decision Rule learns by clinician feedback and is initially configured using information describing the individual treatment response. If the Decision Rule (208) predicts relapse, a flag is sent to the clinician in step (210) (e.g., via text, email, or other form of notification) that indicates recommendation for retreatment, or recommendation for a clinical evaluation to further examine the need for retreatment. In some variations, in addition to or instead of predicting relapse, the Decision Rule (208) predicts the score of a clinician administered inventory such as the MADRS, or predicts whether the score on a clinician administered inventory is above or below a given score, for example a threshold associated with relapse such as a score of 10. In step (214), the clinician may make a decision about re-treatment based on the score of the actual MADRS that is administered when the patient is brought back in, and/or clinician feedback on whether the patient is actually judged to need retreatment. This actual assessment score and/or clinical decision may be provided back to the Decision Rule (208) to help refine the algorithm over time, for example by providing training data for refinement of a machine learning algorithm. If the patient is deemed by the clinician to require re-treatment, the patient may be treated until remission or response, or until a pre-determined number of retreatments (e.g., five days) has been delivered.

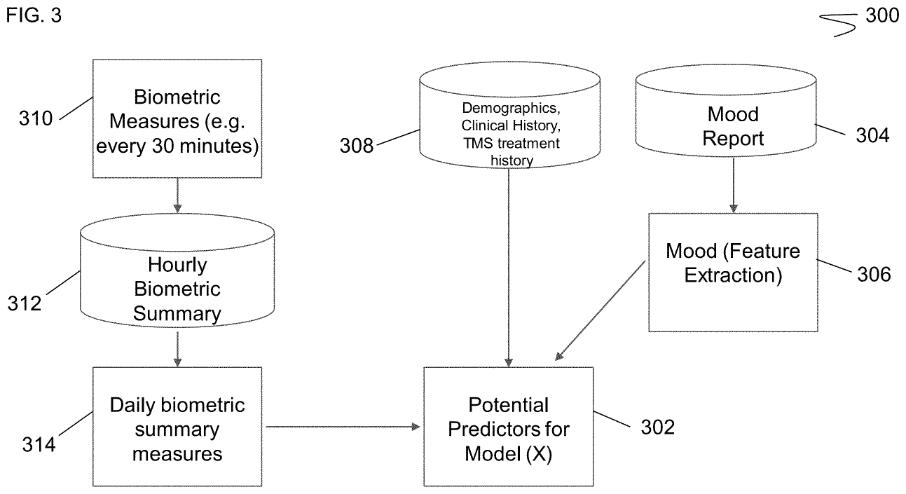

Other variations of the method may include determining a set of potential predictors for model test data such as the test data (X) in FIG. 2. Referring to the flowchart (300) in FIG. 3, data features (data inputs) used to determine the potential predictors (302) for a model test data set include, but are not limited to, patient-reported inventories, patient characteristics, patient demographics, treatment history, and biometric measures. For example, data from a mood report (304) may be obtained and used. The mood report (304) may be a patient self-report of daily mood. The self-report may be a single question consisting of a visual analog scale (100 to −100) where the best possible mood is scored 100 and the worst possible mood is scored −100. The patient may use a mobile application that consists of a sliding scale where the nominal value of the scale is 0. Patient's may use electronic means such as a computer, laptop, mobile phone, tablet computer, smart-watch, wearable device such as a ring, or other means to access an application or website to answer the single-question self-report daily mood inventory. Mood feature extraction (306) may then be performed, and may include extraction of features from daily mood reports and/or their fluctuations, extraction of the overall mood slope across time, as well as time-window mood slopes. The mood reports (304) may be collected over a period of time (e.g., 21 days). The mood features extracted in step (306) may then be added to the set of potential predictors for the model test data (302).

Additional patient data features (data inputs) may be derived from a database (308) including patient demographics (e.g., age, gender, weight), clinical history (e.g., number of disease episodes, previous number of days to relapse, etc.), and TMS treatment history (e.g., average days between treatments, time since last treatment, etc.). Other types of patient data may also be extracted from the database, including brain factors derived from structural magnetic resonance imaging (MRI), functional MRI (fMRI), functional near-infrared spectroscopy (fNIRS), and electroencephalography (EEG).

The potential predictors may further be derived from biometric data of the patient. Biometric data may be collected, for example, using a watch or other wearable device and uploaded to a database. The biometric data may include heart rate, heart rate variability (e.g., heart rate variability at various frequency ranges), body temperature (e.g., taking average temperature and/or fluctuations of body temperature over a period of time), skin conductivity, activity measured, e.g., by an accelerometer, actigraphy, and combinations thereof. The biometric data may be collected periodically, for example every 30 minutes (310), and then compiled into an hourly biometric summary (312), from which a daily biometric summary (314) may then be created. The daily biometric summary (314) may be included as part of the set of potential predictors for model test data (302).

The Montgomery-Asberg Depression Rating Scale (MADRS) is a widely used, ten-item diagnostic questionnaire that clinicians use to measure the severity of depressive episodes in patients. Administration of the MADRS is based on a clinical interview and assesses the following: apparent sadness, reported sadness, inner tension, reduced sleep, reduced appetite, concentration difficulties, lassitude, inability to feel, pessimistic thoughts, and suicidal thoughts. The self-reported MADRS (MADRS-S) is a nine-item inventory that assesses the patient's perceptions of the severity of their own symptoms. The scale consists of 9 items assessing patients' mood, feelings of unease, sleep, appetite, ability to concentrate, initiative, emotional involvement, pessimism, and zest for life.

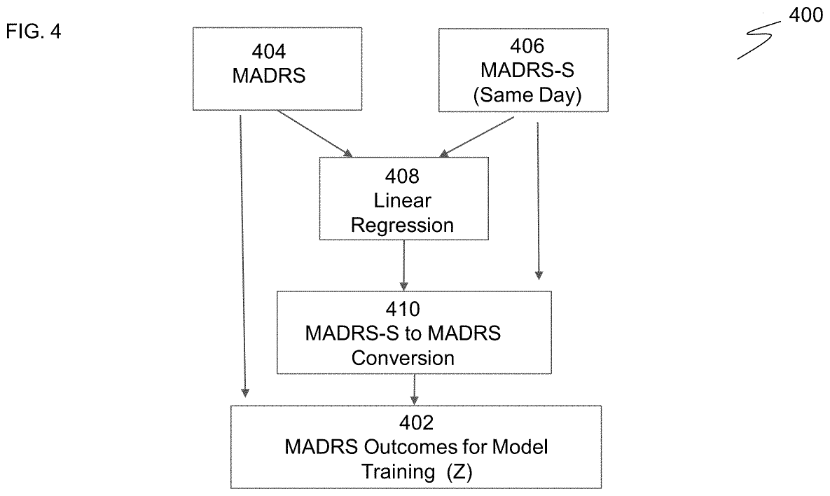

In some variations, a predictive model may be used that provides an estimation of a clinician administered inventory such as the MADRS. For example, referring to FIG. 4, a flowchart (400) illustrating a method for determining outcomes for model training data (402) is shown. In this method, the results from MADRS (404) and MADRS-S(406) completed on the same day are extracted from a database. Linear regression (408) may then be implemented followed by conversion of MARDS-S to MADRS (410). Thereafter, the most recent MADRS and converted MADRS may be used as part of the MADRS Outcomes for Model Training, Z (402).

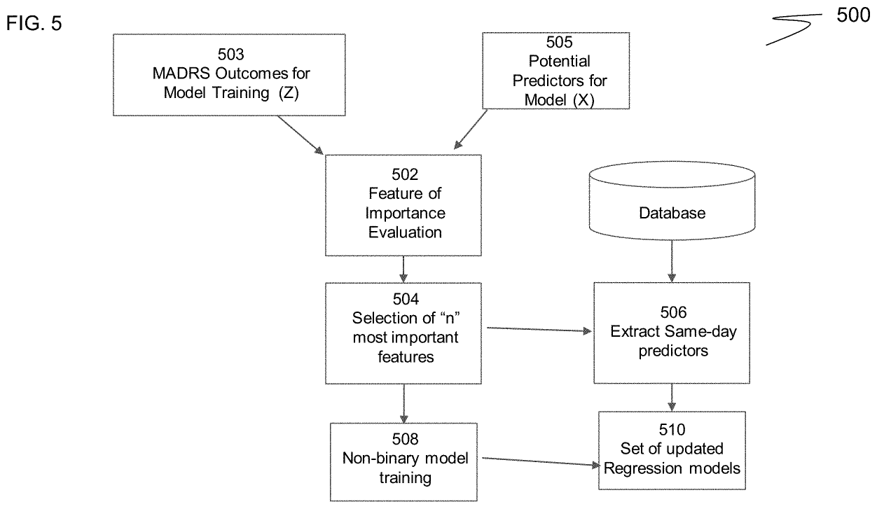

The predictive model may also employ one or more machine learning algorithms. In some variations, the machine learning algorithms utilize regression models. Referring to FIG. 5, a flowchart (500) illustrating an exemplary method for implementing a machine learning approach to extract features and develop a set of regression models is shown. The initial model steps may involve feature reduction. It may be important to reduce the number of features to reduce the chance of overfitting, which is having a model that is useful at predicting training data but not in predicting the test data. In this method, the Feature of Importance Evaluation (502) may analyze features obtained from a MADRS (503) and/or potential predictors (505) (e.g., as determined by the method shown in FIG. 3), and may be performed by implementing an ensemble learning method.

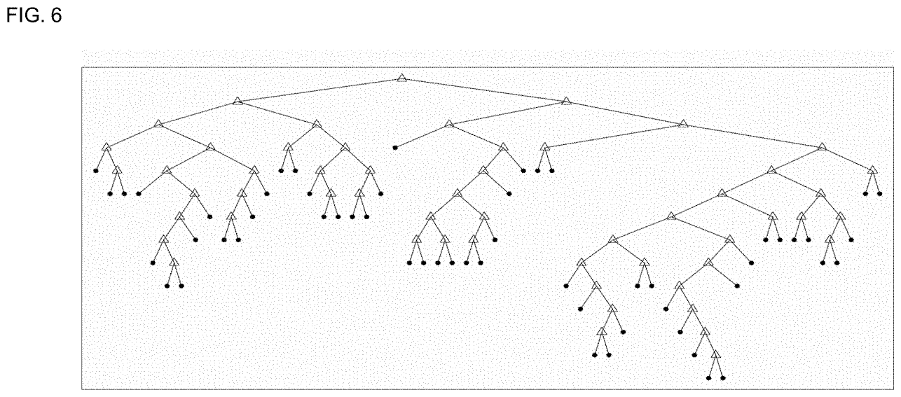

The Feature of Importance may be evaluated using a Random Forest. Random Forest is generally a classifier that contains a number of decision trees on various subsets of a given dataset and takes the average to improve the predictive accuracy. Instead of relying on one decision tree, the Random Forest takes the prediction from each tree and based on the majority of votes of predictions, predicts the final output. The greater number of trees in the forest may lead to higher accuracy and may prevent the problem of overfitting. FIG. 6. is an example of one out of ten decision trees that may be used in the Feature of Importance Evaluation (502) of FIG. 5. In this example, the feature of importance is evaluated using Random Forest in an N by K-fold (e.g., K=5) cross validation framework where N>1 (e.g., number of samples/K) to yield a set of N*K models, each of which is based on a subset of samples of size (K−1)/K and is used to obtain a feature importance metric for each potential predictor based on the recorded contribution of the feature to model the performance. This procedure produces a set of N*K observed importance values for each feature. A positive importance value suggests that a feature may be contributing to the model and the value indicates the extent of contribution. Negative importance suggests that the feature may not be contributing to the model and may even introduce noise that lowers model performance. Referring back to FIG. 5, the most important features (504) may then be extracted and transferred to a set of same-day predictors (506) as well as features used in non-binary training (508). A set of updated regression models (510) may then be generated using inputs from same-day predictors and non-binary model training.

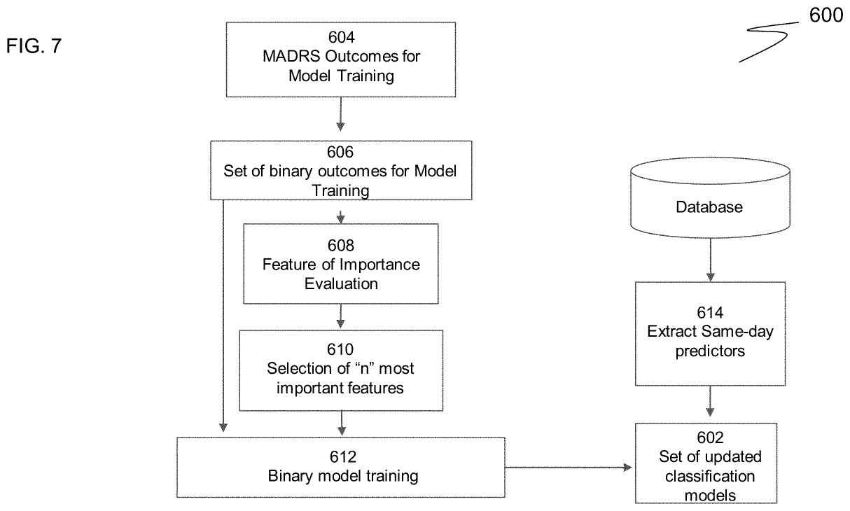

In other variations, the machine learning algorithms utilize classification models. Referring to FIG. 7, a flowchart (600) is provided that shows an exemplary method for implementing a machine learning approach to extract features and develop a set of classification models. The initial step in development of a set of updated classification models (602) may be setting of a threshold value for training data (604) yielding a set of binary outcomes for model training (606). The initial model steps may involve feature reduction. In this method, the Feature of Importance Evaluation (608) may be performed by implementing an ensemble learning method. The most important features (610) may then be extracted and transferred to a set of features used in binary model training (e.g., utilizing support vector machines) (612). Support Vector Machines (SVM) are generally a set of supervised learning methods used for classification, regression, and outlier detection. A SVM may be a supervised machine learning model that uses classification algorithms for two-group classification problems. After giving an SVM model sets of labeled training data for each category, new data may be categorized. A SVM may take the data points and output a hyperplane that creates a decision boundary. A set of updated classification models (602) may then be generated using inputs from same-day predictors (614) and binary model training (612).

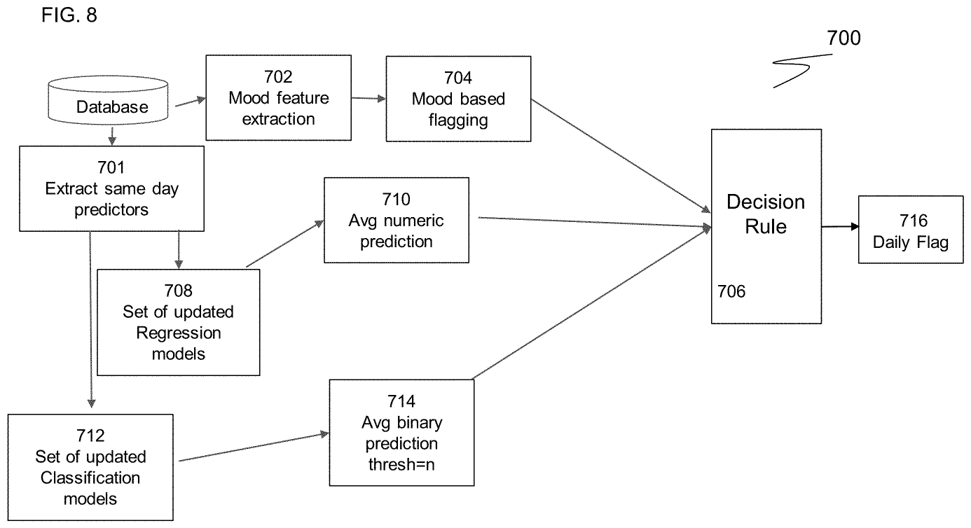

After predicting relapse, maintenance treatment may be performed. For example, FIG. 8 shows a flowchart (700) illustrating an exemplary method for predicting relapse and sending a flag to a clinician to trigger maintenance therapy. In FIG. 8, the mood feature extraction (702) may include features extracted from daily mood reports and their fluctuations, including but not limited to, the overall mood slope across time, as well as time-window mood slopes, e.g. mood reports collected over a period of time (e.g., 21 days). The extracted mood features (702) may then be used to create mood-based flags (704). Criteria for creating a mood-based flag (704) may be a comparison to a patient-specific mood threshold. The mood-based flag (704) may be an input to a decision rule that predicts relapse (706). A set of updated regression models (708) based on same-day predictors (701), e.g., similar to the regression models (510) described in FIG. 5, may then provide data to create an average numeric prediction (710) that may be an input to the decision rule that predicts relapse (706). A set of updated classification models (712) based on same-day predictors (701), e.g., similar to the classification models (602) described in FIG. 7, may then provide data to create one or more average binary predictions (714) where each threshold value is n. Each average binary prediction (714) may be an input to the decision rule that estimates MADRS (706). A daily flag (716) is sent to the physician. For example, the flag or notification may include a report that provides the estimated MADRS information. The clinician may utilize the estimated MADRS to support a decision for retreatment. The report may also include a recommended algorithm for treatment.

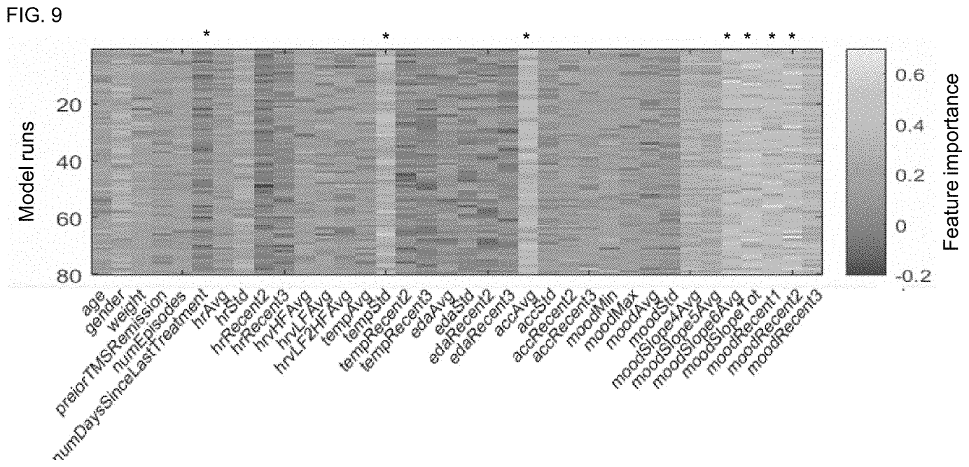

The selection of data features predictive of relapse may be achieved in various ways. For example, FIG. 9 depicts an exemplary feature selection procedure. In FIG. 9, the feature of importance may be calculated using a Random Forest cross validation framework where each row in the heat map (800) corresponds to one model and each column to one potential predictor. Data features with over 80% missing values may be eliminated. The shading scale used in the figure indicates mapping between shade and value of the feature (column) in a given model (row). Features with a contribution that is significantly positive may be selected to be used in the downstream overall model. The procedure may be repeated iteratively after removing the data features having over 80% missing values from the set of predictors.

The data features that may be included in the predictive model include without limitation: (a) age; (b) gender; (c) patient weight; (d) prior TMS remission; (e) number of MDD episodes in a given period such as the patient's lifetime or the current course of maintenance treatment; (f) time such as number of days since the last treatment; (g) heart rate; (h) standard deviation of heart rate; (i) high-frequency power in heart rate variability; (j) average low-frequency power in heart rate variability; (k) ratio of low-frequency to high-frequency power in heart rate variability; (l) body temperature; (m) electrodermal activity; (n) standard deviation of electrodermal activity; (o) accelerometer or actigraphy data; (p) mood inventory minimum value; (q) mood inventory maximum value; (r) mood inventory average value; (s) mood inventory standard deviation; (t) slope of mood inventory value; (u) recent mood; or a combination thereof. In some variations, at least one data feature may be included in the predictive model. In other variations, a plurality of features are included. These features may be calculated across various timescales; for instance, an average value, slope, standard deviation, or other measurement may be calculated or measured across less than one day, one day, two days, three days, four days, five days, six days, seven days, or more. These features may be calculated using various methods; for instance, the variance of a feature may be used instead of the standard deviation of a features. Multiple features of the same type may be included; for example, mood slope calculated over three days, mood slope calculated over four days, mood slope calculated over five days, and/or mood slope calculated over six days. In some variations, features may be continuous data, features that combine (e.g. by summing, multiplication, or other calculation) multiple other features, flags or categorical variables, flags or categorical variables calculated from continuous data, or any other type of data feature.

### EXAMPLES

The following examples are illustrative only, and should not be construed as limiting the disclosure in any way.

### Example 1: Selection of Data Features Predictive of Relapse

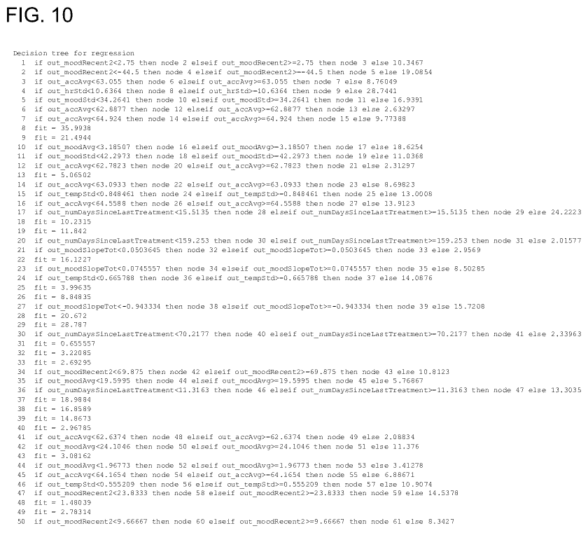

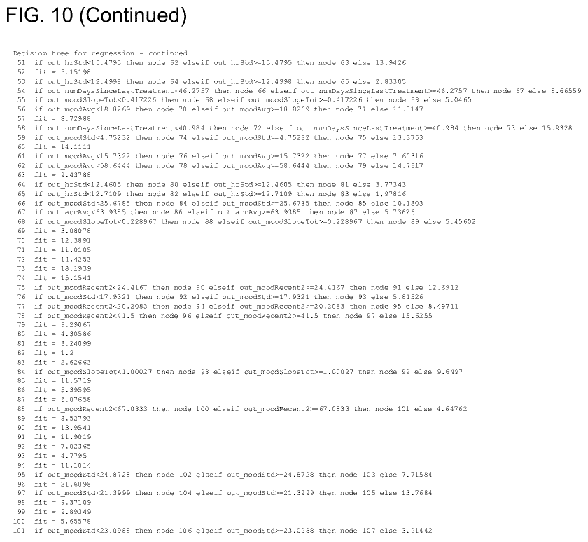

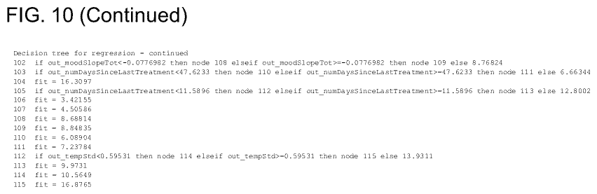

In this example, the feature selection process for a test data set yielded eight factors most predictive of the need for maintenance treatment: slope of mood inventory value (labeled “out_moodSlopeTot”), recent mood inventory value (labeled “out_moodRecent2”), number of days since last treatment (labeled “out_numDaysSinceLastTreatment”), mean acceleration on a wrist-worn device (labeled “out_accAvg”), standard deviation of mood inventory value (labeled “out_moodStd”), standard deviation of body temperature (labeled “out_tempStd”), standard deviation of heart rate (labeled “out_hrStd”), and average mood inventory value (labeled “out_moodAvg”). Referring to FIG. 10, an exemplary decision tree is shown fit to clinical, mood, and MADRS data using these predictive factors. By stepping through the decision tree algorithm, a prediction of MADRS score can be generated. It is understood that data features other than those employed in this example may be used. For example, the decision tree may use any of the other data features previously described herein in whole or in part; use other combinations of data features; use a subset or a superset of these data features; and/or use cut points (i.e., values that predictive features are compared against to determine the next node to travel to in the decision tree), which are partially or substantially different from the cut points shown in FIG. 10.

Additional examples of combinations or sets of data features used in a decision tree may include, without limitation: (a) recent mood inventory value and slope of mood inventory value; (b) recent mood inventory value, slope of mood inventory value, and number of days since last treatment; (c) recent mood inventory value, slope of mood inventory value, and mean acceleration on a wrist-worn device; (d) recent mood inventory value, slope of mood inventory value, number of days since last treatment, standard deviation of mood inventory value, and mean acceleration on a wrist-worn device; (e) recent mood inventory value, slope of mood inventory value, and standard deviation of body temperature; (f) recent mood inventory value, slope of mood inventory value, number of days since last treatment, standard deviation of mood inventory value, and standard deviation of body temperature; (g) recent mood inventory value, slope of mood inventory value, and standard deviation of heart rate; (h) recent mood inventory value, slope of mood inventory value, number/of days since last treatment, standard deviation of mood inventory value, and standard deviation of heart rate; (i) standard deviation of heart rate, mean acceleration on a wrist-worn device, and standard deviation of body temperature; (j) standard deviation of heart rate and mean acceleration on a wrist-worn device; (k) standard deviation of heart rate and standard deviation of body temperature; and (l) standard deviation of body temperature and mean acceleration on a wrist-worn device. It is understood that other combinations or sets of data features may be used in the decision trees. In some variations, the algorithm(s) described herein may be used to monitor symptoms of depression in order to provide value to a patient, such as understanding of one's own body and mind, without or in addition to directly triggering treatment. In other variations, the data features may be used as predictive factors in a model other than or in addition to a decision tree model, for instance a generalized linear model, support vector machine, or other machine learning model described herein in whole or in part.

In further variations, decision tree algorithms may include decision trees where the first branching step may be determined based on recent mood inventory value, for instance mood inventory collected via a visual analogue scale from 0 to 10 points and with a cut point for the branching step between 0 and 9 such as 2.5, 2.75, 3.0, 3.5, or 4.0. In additional variations, decision tree algorithms may include decision trees where a first, second, or third branching step may be determined based on standard deviation of body temperature, for instance with a cut point for the branching step between 0.8 and 0.9 degrees, between 0.7 and 0.8 degrees, between 0.6 and 0.7 degrees, between 0.50 and 0.60 degrees, between 0.40 and 0.5 degrees, between 0.30 and 0.4 degrees, between 0.2 and 0.3 degrees, between 0.1 and 0.2 degrees, and between 0.0 degrees and 0.1 degrees. In yet further variations, decision tree algorithms may include decision trees where a first, second, or third branching step may be determined based on standard deviation of heart rate. In some variations, decision tree algorithms may include decision trees where a first, second, or third branching step may be determined based on mean acceleration on a wrist-worn device.

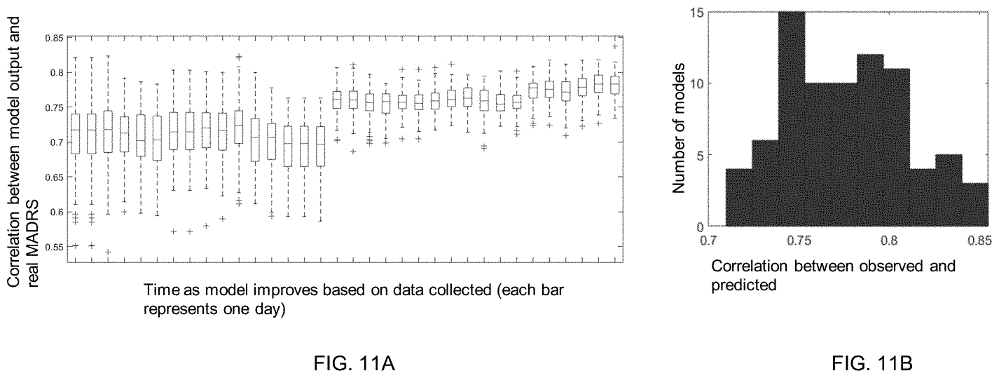

In FIG. 11A, an example bar chart is provided that shows the distribution of the correlation coefficients between predicted values of the MADRS based on the predictive factors mentioned above (and shown in FIG. 10) and real, clinically-measured values of the MADRS. Additionally, FIG. 11B provides an example box-and-whisker plot that illustrates the predictive power of a decision tree model such as the one shown here improving over time as more data are collected.

### Example 2: Clinical Example of Relapse Prediction

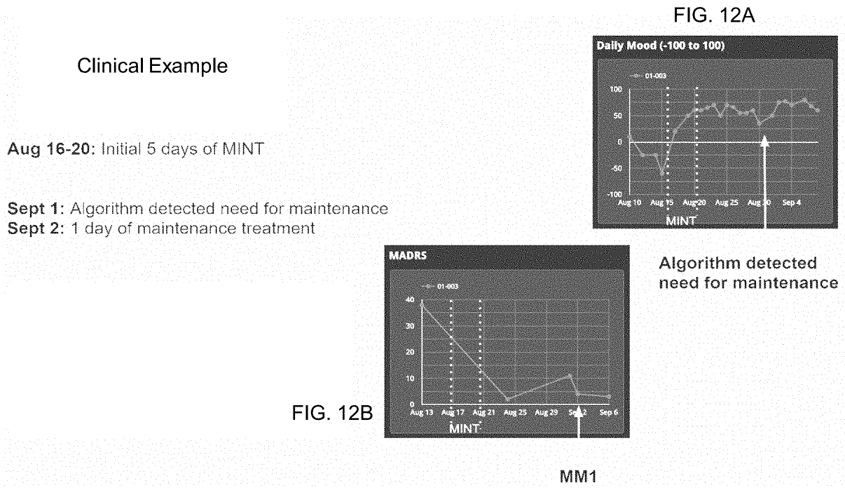

Referring to FIGS. 12A and 12B, a clinical example of a predictive algorithm is provided. The patient was treated daily for 5 days of maintenance therapy. A mood plot showing timecourse of the daily mood report (visual analog scale), collected on a mobile application, is shown in FIG. 12A. The vertical lines illustrate the initial treatment period between August 16^{th} and August 20^{th}. The arrow indicates that the algorithm detected the need for maintenance because the patient's daily mood dropped below a given threshold, showing that the algorithm is capable of early detection of relapse. In this particular case, the patient strongly confirmed that the algorithm had accurately detected early signs of relapse. On the lower plot shown in FIG. 12B, the timecourse of the MADRS is shown, confirming that an upward peak above a score of 10 was observed after the patient was invited back to the clinic, indicating that relapse was occurring. At this point, maintenance treatment was delivered (labeled “MM1”), the mood score lifted, and the MADRS showed that the patient was back in remission.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A system for predicting relapse of a neurological or a psychiatric disorder of a patient comprising:
a device configured to obtain one or more data features from the patient; and
a data module, the data module comprising one or more processors configured to run a machine learning algorithm, wherein the machine learning algorithm is configured to:
analyze the one or more data features;
generate a mood report based on the analyzed one or more data features;
generate a mood plot having a mood threshold predetermined for the patient based on a plurality of mood reports taken over a plurality of neurostimulation treatment sessions; and
predict relapse of the neurological or the psychiatric disorder in the patient based on the mood plot.

2. The system of claim 1, wherein relapse is predicted if the machine learning algorithm determines that the mood plot does not meet the predetermined mood threshold for the patient.

3. The system of claim 1, wherein the psychological disorder is depression, treatment-resistant depression, anxiety, post-traumatic stress disorder (PTSD), obsessive-compulsive disorder (OCD), a substance use disorder, bipolar disorder, or schizophrenia.

4. The system of claim 1, wherein the neurological disorder is Parkinson's disease, essential tremor, stroke, epilepsy, traumatic brain injury, migraine headache, cluster headache, or chronic pain.

5. (canceled)

6. The system of claim 1, wherein the one or more data features comprises mood data.

7. The system of claim 6, wherein the mood data comprises a patient self-report of daily mood using a visual analog scale.

8. The system of claim 6, wherein the mood data comprises psychometric data.

9. The system of claim 8, wherein the psychometric data comprises information relating to mind wandering, anxiety, processing speed, task switching ability, attention, loneliness, or a combination thereof.

10. The system of claim 1, wherein the one or more data features comprises information relating to motor activity.

11. The system of claim 1, wherein the one or more data features comprises information related to heart rate, heart rate variability, electroencephalography, electrogastrography, electrogastroenterography, galvanic skin response, sleep, sweat chloride, neuroimaging, patient demographics, outcome data from an acute treatment, outcome data from a prior maintenance treatment, or a combination thereof.

12. The system of claim 11, wherein the information related to sleep comprises a total duration of sleep, a sleep onset time, a sleep offset time, a sleep cycle duration, a number of sleep cycles per night, sleep movements, sleep vocalizations, or a combination thereof.

13. The system of claim 1, wherein the one or more data features comprises body temperature, a mean body temperature within a given period of time, a fluctuation in body temperature, or a combination thereof.

14. The system of claim 1, wherein the one or more data features comprises information estimated from a clinician administered inventory.

15. The system of claim 1, wherein the device comprises a computer, a laptop, a tablet computer, a mobile phone, a smart-watch, or a ring.

16. The system of claim 1, further comprising a treatment device.

17. The system of claim 16, wherein the treatment device comprises a transcranial magnetic stimulation coil.

18. The system of claim 1, wherein the machine learning algorithm is further configured to recommend a treatment schedule to minimize relapse of the neurological or the psychiatric disorder.

19. A method for predicting relapse of a neurological or a psychiatric disorder of a patient comprising:
inputting one or more data features from the patient into a predictive model for the neurological or the psychiatric disorder;
applying a machine learning algorithm to the one or more data features to generate a mood report and a mood plot, wherein the mood plot has a mood threshold predetermined for the patient based on a plurality of mood reports taken over a plurality of neurostimulation treatment sessions; and
predicting relapse of the neurological or the psychiatric disorder in the patient based on the mood plot.

20.-30. (canceled)

31. The method of claim 19, wherein the one or more data features comprises body temperature, a mean body temperature within a given period of time, a fluctuation in body temperature, or a combination thereof.

32.-33. (canceled)

34. The method of claim 19, further comprising delivering neurostimulation therapy.

35.-40. (canceled)
